(19) United States
(12) Patent Application Publication
(10) Pub. No.: US 2021/0169430 A1
(43) Pub. Date: Jun. 10, 2021

(54) APPARATUS AND METHOD FOR IMAGING AN OBJECT USING RADIATION

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 17/179,483

(22) Filed: Feb. 19, 2021

Related U.S. Application Data
(63) Continuation of application No. PCT/CN2018/104593, filed on Sep. 7, 2018.

(51) Int. Cl. A61B 6/00; A61B 6/06

(52) U.S. Cl. CPC A61B 6/4452; A61B 6/4266; A61B 6/502; A61B 6/4233; A61B 6/5241; A61B 6/4028; A61B 6/06

(57) ABSTRACT

Disclosed herein an apparatus comprising: a radiation source configured to produce a beam of radiation toward an object; an image sensor comprising a plurality of radiation detectors spaced apart from one another. The image sensor is configured to move between a first position and a second position, relative to the object. The radiation source is configured to move along a path relative to the object. Disclosed herein is a method comprising: positioning an image sensor at a first position relative to an object, the image sensor comprising a plurality of radiation detectors spaced apart from one another; capturing a first set of images of the object, by using the radiation detectors and with a beam of radiation from a radiation source, while moving the radiation source among a first plurality of positions on a path, relative to the object; positioning the image sensor at a second position relative to the object; capturing a second set of images of the object, by using the radiation detectors and with the beam of radiation, while moving the radiation source among a second plurality of positions on the path, relative to the object.

### BACKGROUND

Image sensors based on radiation detectors may be devices used to measure the flux, spatial distribution, spectrum or other properties of radiation such as X-rays. These image sensors may be used for many applications. One important application is medical imaging in which the internal structure of a non-uniformly composed and opaque object such as the human body may be revealed.

### SUMMARY

Disclosed herein is an apparatus comprising: a radiation source configured to produce a beam of radiation toward an object; an image sensor comprising a plurality of radiation detectors spaced apart from one another. The image sensor is configured to move between a first position and a second position, relative to the object. The radiation source is configured to move along a path relative to the object.

According to an embodiment, the image sensor is configured to capture a first set of images of the object, by using the radiation detectors and with the beam of radiation, while the image sensor is at the first position and the radiation source is respectively at a first plurality of positions on the path. The image sensor is configured to capture a second set of images of the object, by using the radiation detectors and with the beam of radiation, while the image sensor is at the second position and the radiation source is respectively at a second plurality of positions on the path.

According to an embodiment, the first plurality of positions on the path and the second plurality of positions on the path are the same.

According to an embodiment, the apparatus further comprises a processor configured to stitch at least one image in the first set and at least one image in the second set.

According to an embodiment, the apparatus further comprises a processor configured to determine a three-dimensional structure of the object based on the first set of images or the second set of images.

According to an embodiment, the object is a breast of a human.

According to an embodiment, the radiation source is configured to rotate with respect to the object, while the radiation source moves along the path.

According to an embodiment, the path is an arc around the object.

According to an embodiment, the image sensor comprises a collimator with a plurality of radiation transmitting zones and a radiation blocking zone. The radiation blocking zone is configured to block radiation that would otherwise incident on a dead zone of the image sensor, and the radiation transmitting zones are configured to transmit at least a portion of radiation that would incident on active areas of the image sensor.

According to an embodiment, at least some of the plurality of radiation detectors are arranged in staggered rows.

According to an embodiment, wherein radiation detectors in a same row are uniform in size; wherein a distance between two neighboring radiation detectors in a same row is greater than a width of one radiation detector in the same row in an extending direction of the row and is less than twice that width.

According to an embodiment, at least some of the plurality of radiation detectors are rectangular in shape.

According to an embodiment, at least some of the plurality of radiation detectors are hexagonal in shape.

According to an embodiment, the beam of radiation is a divergent beam of radiation.

According to an embodiment, the radiation is X-ray.

According to an embodiment, at least one of the plurality of radiation detectors comprises a radiation absorption layer and an electronics layer. The radiation absorption layer comprises an electrode. The electronics layer comprises an electronic system. The electronic system comprises: a first voltage comparator configured to compare a voltage of the electrode to a first threshold, a second voltage comparator configured to compare the voltage to a second threshold, a counter configured to register a number of particles of radiation reaching the radiation absorption layer, and a controller. The controller is configured to start a time delay from a time at which the first voltage comparator determines that an absolute value of the voltage equals or exceeds an absolute value of the first threshold. The controller is configured to activate the second voltage comparator during the time delay. The controller is configured to cause the number registered by the counter to increase by one, if the second voltage comparator determines that an absolute value of the voltage equals or exceeds an absolute value of the second threshold.

According to an embodiment, the electronic system further comprises an integrator electrically connected to the electrode, wherein the integrator is configured to collect charge carriers from the electrode.

According to an embodiment, the controller is configured to activate the second voltage comparator at a beginning or expiration of the time delay.

According to an embodiment, the electronic system further comprises a voltmeter, wherein the controller is configured to cause the voltmeter to measure the voltage upon expiration of the time delay.

According to an embodiment, the controller is configured to determine an energy of particles of radiation based on a value of the voltage measured upon expiration of the time delay.

According to an embodiment, the controller is configured to connect the electrode to an electrical ground.

According to an embodiment, a rate of change of the voltage is substantially zero at expiration of the time delay.

According to an embodiment, a rate of change of the voltage is substantially non-zero at expiration of the time delay.

Disclosed herein is a method comprising: positioning an image sensor at a first position relative to an object, the image sensor comprising a plurality of radiation detectors spaced apart from one another; capturing a first set of images of the object, by using the radiation detectors and with a beam of radiation from a radiation source, while moving the radiation source among a first plurality of positions on a path, relative to the object; positioning the image sensor at a second position relative to the object; capturing a second set of images of the object, by using the radiation detectors and with the beam of radiation, while moving the radiation source among a second plurality of positions on the path, relative to the object.

According to an embodiment, the first plurality of positions on the path and the second plurality of positions on the path are the same.

According to an embodiment, the method further comprises stitching at least one image in the first set and at least one image in the second set.

According to an embodiment, the method further comprises determining a three-dimensional structure of the object based on the first set of images or the second set of images.

According to an embodiment, the object is a breast of a human.

According to an embodiment, moving the radiation source comprises rotating the radiation source with respect to the object.

According to an embodiment, the path is an arc around the object.

According to an embodiment, the image sensor comprises a collimator with a plurality of radiation transmitting zones and a radiation blocking zone. The radiation blocking zone is configured to block radiation that would otherwise incident on a dead zone of the image sensor, and the radiation transmitting zones are configured to transmit at least a portion of radiation that would incident on active areas of the image sensor.

According to an embodiment, at least some of the plurality of radiation detectors are arranged in staggered rows.

According to an embodiment, radiation detectors in a same row are uniform in size; wherein a distance between two neighboring radiation detectors in a same row is greater than a width of one radiation detector in the same row in an extending direction of the row and is less than twice that width.

According to an embodiment, at least some of the plurality of radiation detectors are rectangular in shape.

According to an embodiment, at least some of the plurality of radiation detectors are hexagonal in shape.

According to an embodiment, the beam of radiation is a divergent beam of radiation.

According to an embodiment, the radiation is X-ray.

According to an embodiment, at least one of the plurality of radiation detectors comprises a radiation absorption layer and an electronics layer. The radiation absorption layer comprises an electrode. The electronics layer comprises an electronic system. The electronic system comprises: a first voltage comparator configured to compare a voltage of the electrode to a first threshold, a second voltage comparator configured to compare the voltage to a second threshold, a counter configured to register a number of particles of radiation reaching the radiation absorption layer, and a controller. The controller is configured to start a time delay from a time at which the first voltage comparator determines that an absolute value of the voltage equals or exceeds an absolute value of the first threshold. The controller is configured to activate the second voltage comparator during the time delay. The controller is configured to cause the number registered by the counter to increase by one, if the second voltage comparator determines that an absolute value of the voltage equals or exceeds an absolute value of the second threshold.

According to an embodiment, the electronic system further comprises an integrator electrically connected to the electrode, wherein the integrator is configured to collect charge carriers from the electrode.

According to an embodiment, the controller is configured to activate the second voltage comparator at a beginning or expiration of the time delay.

According to an embodiment, the electronic system further comprises a voltmeter, wherein the controller is configured to cause the voltmeter to measure the voltage upon expiration of the time delay.

According to an embodiment, the controller is configured to determine an energy of particles of radiation based on a value of the voltage measured upon expiration of the time delay.

According to an embodiment, the controller is configured to connect the electrode to an electrical ground.

According to an embodiment, a rate of change of the voltage is substantially zero at expiration of the time delay.

According to an embodiment, a rate of change of the voltage is substantially non-zero at expiration of the time delay.

### BRIEF DESCRIPTION OF FIGURES

FIG. 1 schematically shows an apparatus, according to an embodiment.

FIG. 2A schematically shows a cross-sectional view of a radiation detector of an image sensor of the apparatus, according to an embodiment.

FIG. 2B schematically shows a detailed cross-sectional view of the radiation detector, according to an embodiment.

FIG. 2C schematically shows an alternative detailed cross-sectional view of the radiation detector, according to an embodiment.

FIG. 3 schematically shows that the radiation detector may have an array of pixels, according to an embodiment.

FIG. 4A schematically shows a top view of a package including the radiation detector and a printed circuit board (PCB).

FIG. 4B schematically shows a cross-sectional view of the image sensor, where a plurality of the packages of FIG. 4A are mounted to another PCB.

FIG. 5 schematically shows a collimator of the image sensor, according to an embodiment.

FIG. 6 schematically shows that the image sensor may capture multiple sets of images when it is respectively at multiple positions relative to the object.

FIG. 7A schematically shows the image of an object can be formed by stitching images of multiple different portions of an object, according to an embodiment.

FIG. 7B schematically shows the image of an object can be formed by stitching images of multiple different portions of an object, according to an embodiment.

FIG. 8A-FIG. 8C schematically show arrangements of the detectors in the image sensor, according to some embodiments.

FIG. 9 schematically shows detectors that are hexagonal in shape, according to an embodiment.

FIG. 10 schematically shows the flowchart of a method, according to an embodiment.

FIG. 11A and FIG. 11B each show a component diagram of an electronic system of the detector in FIG. 2A, FIG. 2B and FIG. 2C, according to an embodiment.

FIG. 12 schematically shows a temporal change of the electric current flowing through an electrode (upper curve) of a diode or an electrical contact of a resistor of a radiation absorption layer exposed to radiation, the electric current caused by charge carriers generated by a particle of radiation incident on the radiation absorption layer, and a corresponding temporal change of the voltage of the electrode (lower curve), according to an embodiment.

FIG. 13 schematically shows a temporal change of the electric current flowing through the electrode (upper curve) caused by noise (e.g., dark current), and a corresponding temporal change of the voltage of the electrode (lower curve), in the electronic system operating in the way shown in FIG. 12, according to an embodiment.

FIG. 14 schematically shows a temporal change of the electric current flowing through an electrode (upper curve) of the radiation absorption layer exposed to radiation, the electric current caused by charge carriers generated by a particle of radiation incident on the radiation absorption layer, and a corresponding temporal change of the voltage of the electrode (lower curve), when the electronic system operates to detect incident particles of radiation at a higher rate, according to an embodiment.

FIG. 15 schematically shows a temporal change of the electric current flowing through the electrode (upper curve) caused by noise (e.g., dark current), and a corresponding temporal change of the voltage of the electrode (lower curve), in the electronic system operating in the way shown in FIG. 14, according to an embodiment.

FIG. 16 schematically shows a temporal change of the electric current flowing through the electrode (upper curve) caused by charge carriers generated by a series of particles of radiation incident on the radiation absorption layer, and a corresponding temporal change of the voltage of the electrode, in the electronic system operating in the way shown in FIG. 14 with RST expires before tₑ, according to an embodiment.

### DETAILED DESCRIPTION

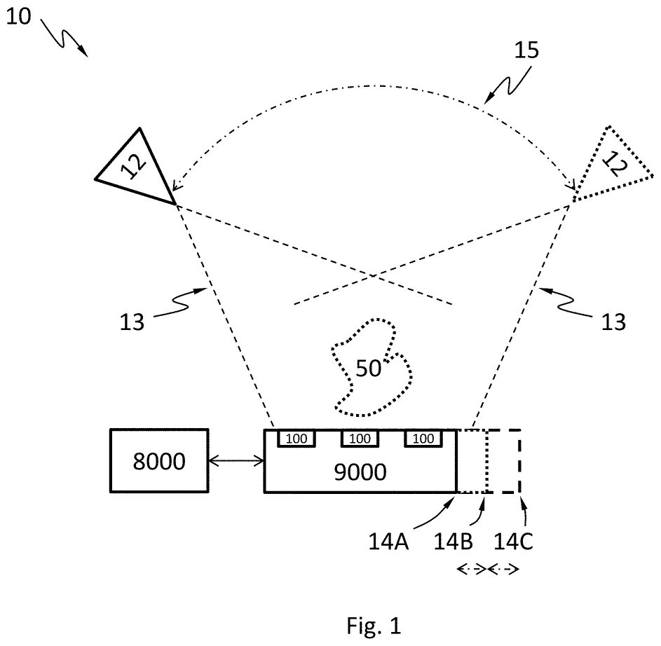

FIG. 1 schematically shows an apparatus 10, according to an embodiment. The apparatus 10 comprises a radiation source 12 and an image sensor 9000. The radiation source 12 produces a beam 13 of radiation toward an object 50. The beam 13 may be a divergent beam, a convergent beam, a parallel beam or another suitable beam. The radiation from the radiation source 12 may be X-ray or another suitable radiation such as gamma ray. The object 50 may be a human organ or tissue. For example, the object 50 may be a breast of a human. The image sensor 9000 has a plurality of radiation detectors 100 spaced apart from one another. Here, two radiation detectors 100 being “spaced apart” means that a portion of the dead zone of the image sensor 9000 is between the two radiation detectors. The term “dead zone” is explained below. The image sensor 9000 is able to move between multiple positions (e.g., between a first position 14A and a second position 14B, or the first position 14A, the second position 14B and a third position 14C) relative to the object 50. The multiple positions (e.g., the first position 14A, the second position 14B and the third position 14C) may or may not be on the same straight line. Namely, when the image sensor 9000 move between the multiple positions, the image sensor 9000 may move along different directions at different time. The radiation source 12 is able to move along a path 15 relative to the object 50. The radiation source 12 may rotate with respect to the object 50 while it moves along the path 15. The path 15 may be an arc around the object 50. The center of the arc may be on the object 50, between the object 50 and the image sensor 9000, on the image sensor 9000, or on an opposite side of the image sensor 9000 with respect to the object 50.

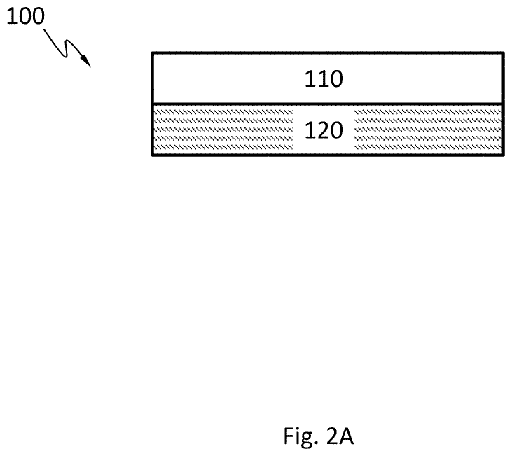

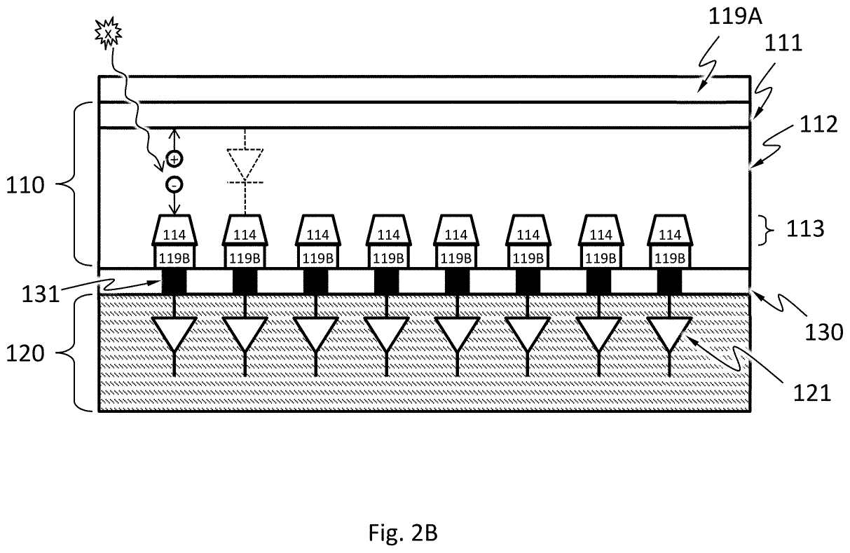

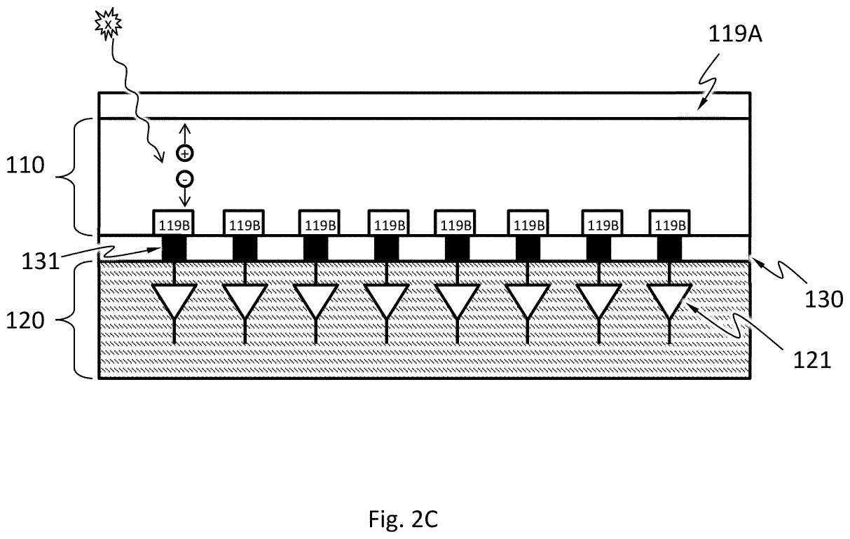

FIG. 2A schematically shows a cross-sectional view of one radiation detector 100 of the image sensor 9000, according to an embodiment. The radiation detector 100 may include a radiation absorption layer 110 and an electronics layer 120 (e.g., an ASIC) for processing or analyzing electrical signals incident radiation generates in the radiation absorption layer 110. In an embodiment, the radiation detector 100 does not comprise a scintillator. The radiation absorption layer 110 may include a semiconductor material such as silicon, germanium, GaAs, CdTe, CdZnTe, or a combination thereof. The semiconductor may have a high mass attenuation coefficient for the radiation produced by the radiation sources in the apparatus 10.

As shown in a detailed cross-sectional view of the radiation detector 100 in FIG. 2B, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes having the first doped region 111 as a shared electrode. The first doped region 111 may also have discrete portions.

When a particle of radiation hits the radiation absorption layer 110 including diodes, the particle of radiation may be absorbed and generate one or more charge carriers by a number of mechanisms. A particle of radiation may generate 10 to 100000 charge carriers. The charge carriers may drift to the electrodes of one of the diodes under an electric field. The field may be an external electric field. Electrical contact 119A is electrically connected with the first doped region 111. Electrical contact 119B may include discrete portions each of which is electrically connected with the discrete regions 114. In an embodiment, the charge carriers may drift in directions such that the charge carriers generated by a single particle of radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of radiation incident therein flow to the discrete region 114. Namely, less than 2%, less than 1%, less than 0.1%, or less than 0.01% of these charge carriers flow beyond the pixel.

As shown in an alternative detailed cross-sectional view of the radiation detector 100 in FIG. 2C, according to an embodiment, the radiation absorption layer 110 may include a resistor of a semiconductor material such as, silicon, germanium, GaAs, CdTe, CdZnTe, or a combination thereof, but does not include a diode. The semiconductor may have a high mass attenuation coefficient for the radiation produced by the radiation source 12.

When a particle of radiation hits the radiation absorption layer 110 including a resistor but not diodes, it may be absorbed and generate one or more charge carriers by a number of mechanisms. A particle of radiation may generate 10 to 100000 charge carriers. The charge carriers may drift to the electrical contacts 119A and 119B under an electric field. The field may be an external electric field. The electrical contact 119B includes discrete portions. In an embodiment, the charge carriers may drift in directions such that the charge carriers generated by a single particle of radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of radiation incident around the footprint of one of these discrete portions of the electrical contact 119B are not substantially shared with another of these discrete portions of the electrical contact 119B. A pixel 150 associated with a discrete portion of the electrical contact 119B may be an area around the discrete portion in which substantially all (more than 98%, more than 99.5%, more than 99.9% or more than 99.99% of) charge carriers generated by a particle of radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

The electronics layer 120 may include an electronic system 121 suitable for processing or interpreting signals generated by particles of radiation incident on the radiation absorption layer 110. The electronic system 121 may include an analog circuitry such as a filter network, amplifiers, integrators, and comparators, or a digital circuit such as a microprocessor and a memory. The electronic system 121 may include components shared by the pixels or components dedicated to a single pixel. For example, the electronic system 121 may include an amplifier dedicated to each pixel and a microprocessor shared among all the pixels. The electronic system 121 may be electrically connected to the pixels by vias 131. Space among the vias may be filled with a filler material 130, which may increase the mechanical stability of the connection of the electronics layer 120 to the radiation absorption layer 110. Other bonding techniques are possible to connect the electronic system 121 to the pixels without using vias.

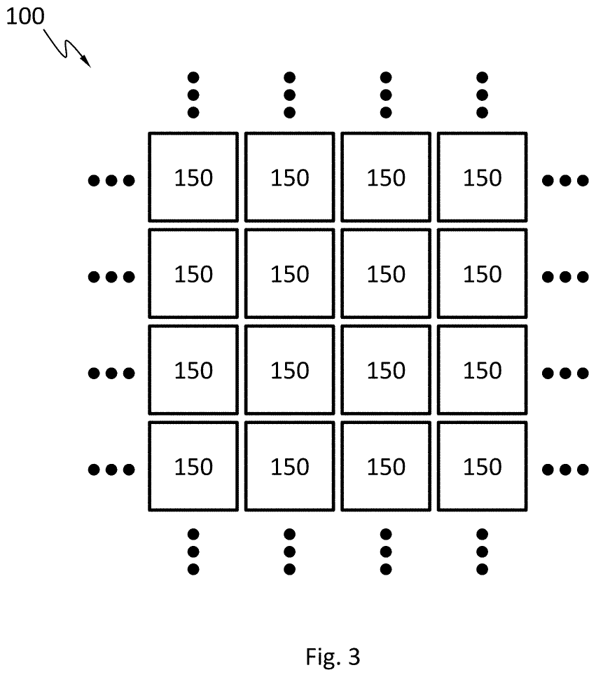

FIG. 3 schematically shows that the radiation detector 100 may have an array of pixels 150. The array may be a rectangular array, a honeycomb array, a hexagonal array or any other suitable array. Each pixel 150 may be configured to detect a particle of radiation incident thereon, measure the energy of the particle of radiation, or both. For example, each pixel 150 may be configured to count numbers of particles of radiation incident thereon whose energy falls in a plurality of bins, within a period of time. All the pixels 150 may be configured to count the numbers of particles of radiation incident thereon within a plurality of bins of energy within the same period of time. Each pixel 150 may have its own analog-to-digital converter (ADC) configured to digitize an analog signal representing the energy of an incident particle of radiation into a digital signal. The ADC may have a resolution of 10 bits or higher. Each pixel 150 may be configured to measure its dark current, such as before or concurrently with each particle of radiation incident thereon. Each pixel 150 may be configured to deduct the contribution of the dark current from the energy of the particle of radiation incident thereon. The pixels 150 may be configured to operate in parallel. For example, when one pixel 150 measures an incident particle of radiation, another pixel 150 may be waiting for a particle of radiation to arrive. The pixels 150 may be but do not have to be individually addressable.

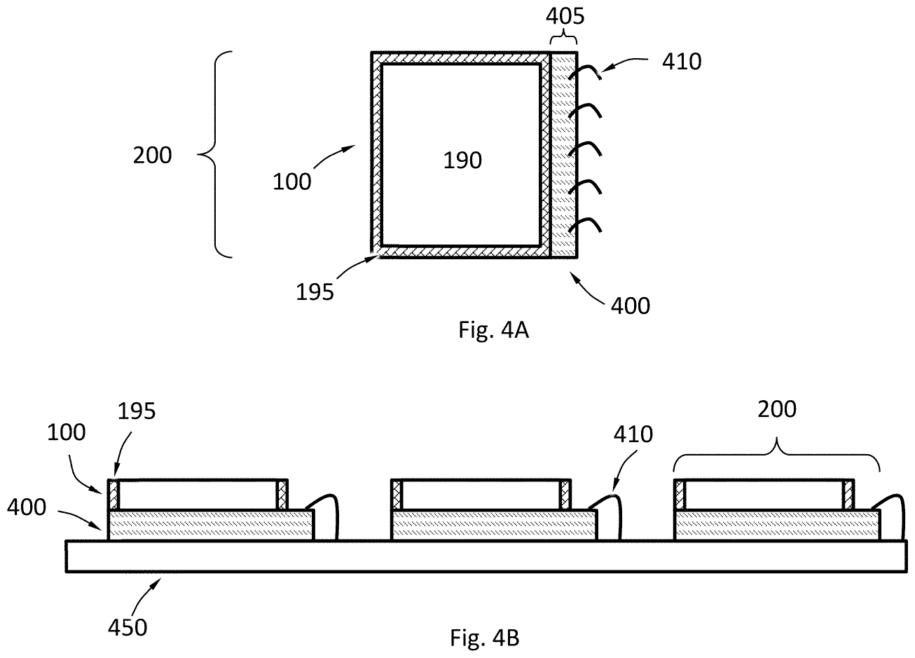

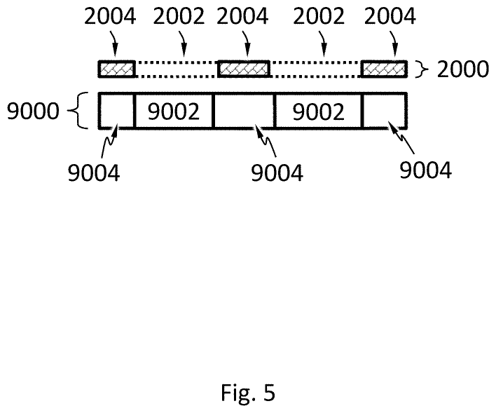

The radiation detectors 100 of the image sensors 9000 may be arranged in any suitable fashion. FIG. 4A and FIG. 4B show an example of the arrangement of the radiation detectors 100 in the image sensor 9000. One or more of the radiation detectors 100 may be mounted on a printed circuit board (PCB) 400. The term “PCB” as used herein is not limited to a particular material. For example, a PCB may include a semiconductor. The radiation detector 100 is mounted to the PCB 400. The wiring between the radiation detectors 100 and the PCB 400 is not shown for the sake of clarity. The PCB 400 and the radiation detectors 100 mounted thereon may be called a package 200. The PCB 400 may have an area not covered by the radiation detectors 100 (e.g., an area for accommodating bonding wires 410). Each of the radiation detector 100 may have an active area 190, which is where the pixels 150 are located. Each of the radiation detector 100 may have a perimeter zone 195 near the edges. The perimeter zone 195 has no pixels and particles of radiation incident on the perimeter zone 195 are not detected.

FIG. 4B schematically shows that the image sensor 9000 may have a system PCB 450 with multiple packages 200 mounted on it. The image sensor 9000 may include one or more such system PCBs 450. The electrical connection between the PCBs 400 in the packages 200 and the system PCB 450 may be made by bonding wires 410. In order to accommodate the bonding wires 410 on the PCB 400, the PCB 400 has an area 405 not covered by the radiation detectors 100. In order to accommodate the bonding wires 410 on the system PCB 450, the packages 200 have gaps in between. The active areas 190 of the radiation detectors 100 in the image sensor 9000 are collectively called the active area of the image sensor 9000. The other areas of the image sensor 9000, radiation incident on which cannot be detected by the image sensor 9000, such as the perimeter zones 195, the area 405 or the gaps between the packages 200, are collectively called the dead zone of the image sensor 9000.

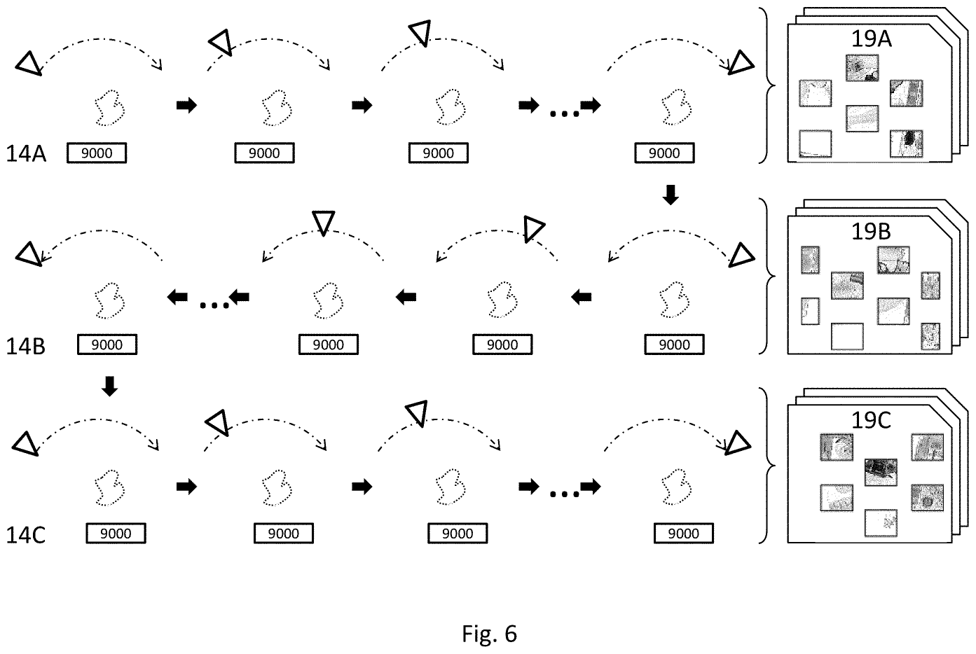

FIG. 5 schematically shows that the image sensor 9000 may comprise a collimator 2000, according to an embodiment. The collimator 2000 comprises a plurality of radiation transmitting zones 2002 and a radiation blocking zone 2004. The radiation blocking zone 2004 substantially blocks radiation that would otherwise incident on the dead zone 9004 of the image sensor 9000, and the radiation transmitting zones 2002 allow at least a portion of radiation that would incident on the active areas 9002 of the image sensor 9000 to pass. The radiation transmitting zones 2002 may be holes through the collimator 2000 and the rest of the collimator 2000 may function as the radiation blocking zone 2004. The material for the collimator 2000, for example, may be lead or other suitable material which can efficiently absorb the radiation produced by the radiation source 12.

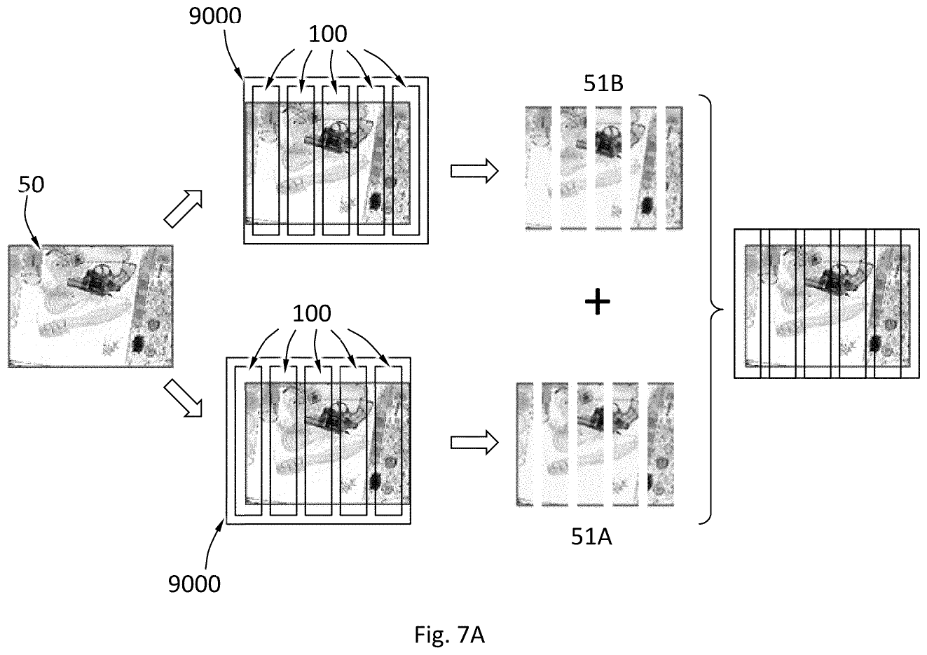

As schematically shown in FIG. 6, the image sensor 9000 may capture a first set 19A of images of the object 50, by using the radiation detectors 100 and with the radiation, while the image sensor 9000 is at the first position 14A and the radiation source 12 is respectively at a first plurality of positions on the path 15. For example, the radiation source 12 may scan continuously along the path 15 and the image sensor 9000 may capture images at multiple points of time during the scan of the radiation source 12. The image sensor 9000 may capture a second set 19B of images of the object 50, by using the radiation detectors 100 and with the radiation, while the image sensor 9000 is at the second position 14B and the radiation source 12 is respectively at a second plurality of positions on the path 15. If the image sensor 9000 can move to additional positions (e.g., the third position 14C), the image sensor 9000 may capture additional sets of images (e.g., the third set of images 19C) of the object 50, by using the radiation detectors 100 and with the radiation, while the image sensor 9000 is respectively at the additional positions and the radiation source 12 is respectively at a plurality of positions on the path 15. The first plurality of positions on the path 15 and the second plurality of positions on the path 15 may be the same. For example, the first set 19A and the second set 19B may each contain an image that was captured when the radiation source 12 is at the same position along the path 15 relative to the object 50.

As shown in FIG. 1, the apparatus 10 may have a processor 8000 that can stitch at least one image in the first set 19A and at least one image in the second set 19B. For example, the at least one image in the first set 19A and the at least one image in the second set 19B may be captured when the radiation source 12 is at the same position along the path 15 relative to the object 50. The processor 8000 may be able to determine a three-dimensional structure of the object 50 based on the first set 19A of images, the second set 19B of images, or both. A suitable algorithm such as the Fourier-Domain Reconstruction Algorithm, the Back Projection Algorithm, the Iterative Reconstruction Algorithm, and the Fan-Beam Reconstruction may be used to calculate the three-dimensional structure of the object 50.

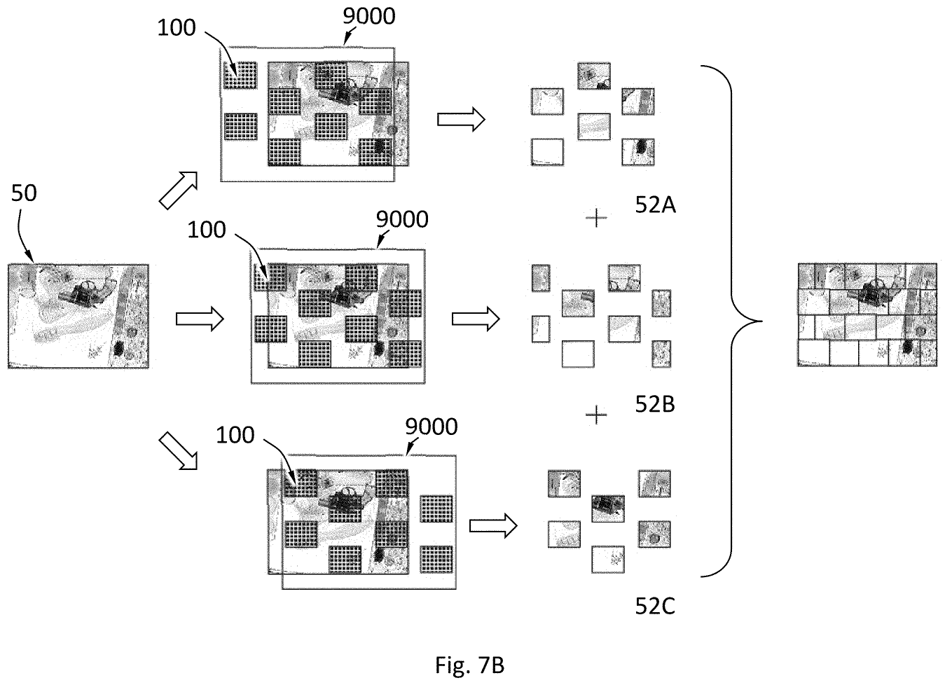

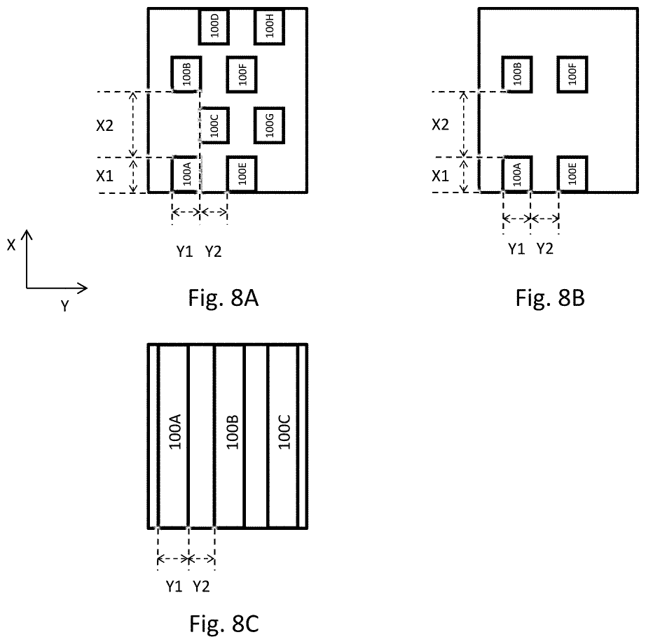

In an example schematically shown in FIG. 7A, image 51A is one in the first set 19A of images; image 51B is one in the second set 19B of images. The images 51A and 51B may be captured when the radiation source 12 was at the same position along the path 15. An image of the object 50 may be formed by stitching the images 51A and 51B (e.g., using the processor 8000).

In an example schematically shown in FIG. 7B, image 52A is one in the first set 19A of images; image 52B is one in the second set 19B of images; and image 52C is one in the third set of images 19C. The images 52A, 52B and 52C may be captured when the radiation source 12 was at the same position along the path 15. An image of the object 50 may be formed by stitching the images 52A, 52B and 52C (e.g., using the processor 8000).

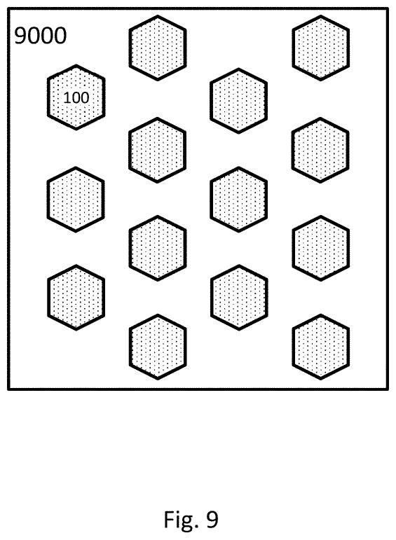

The radiation detectors 100 may be arranged in a variety of ways in the image sensor 9000. FIG. 8A schematically shows one arrangement, according to an embodiment, where the radiation detectors 100 are arranged in staggered rows. For example, radiation detectors 100A and 100B are in the same row, aligned in the Y direction, and uniform in size; radiation detectors 100C and 100D are in the same row, aligned in the Y direction, and uniform in size. Radiation detectors 100A and 100B are staggered in the X direction with respect to radiation detectors 100C and 100D. According to an embodiment, a distance X2 between two neighboring radiation detectors 100A and 100B in the same row is greater than a width X1 (i.e., dimension in the X direction, which is the extending direction of the row) of one detector in the same row and is less than twice the width X1. Radiation detectors 100A and 100E are in a same column, aligned in the X direction, and uniform in size; a distance Y2 between two neighboring radiation detectors 100A and 100E in the same column is less than a width Y1 (i.e., dimension in the Y direction) of one detector in the same column.

FIG. 8B schematically shows another arrangement, according to an embodiment, where the radiation detectors 100 are arranged in a rectangular grid. For example, the radiation detectors 100 may include radiation detectors 100A, 100B, 100E and 100F as arranged exactly in FIG. 8A, without radiation detectors 100C, 100D, 100G, or 100H in FIG. 8A.

Other arrangements may also be possible. For example, in FIG. 8C, the radiation detectors 100 may span the whole width of the image sensor 9000 in the X-direction, with a distance Y2 between two neighboring radiation detectors 100 being less than a width of one detector Y1.

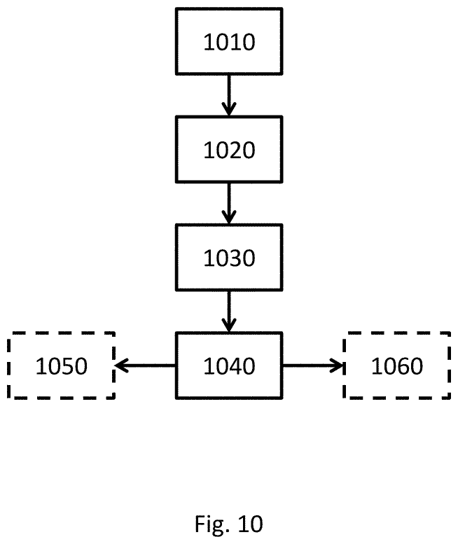

The radiation detectors 100 in the image sensor 9000 have any suitable sizes and shapes. According to an embodiment (e.g., in FIG. 8A-FIG. 8C), at least some of the radiation detectors 100 are rectangular in shape. According to an embodiment, as shown in FIG. 9, at least some of the radiation detectors are hexagonal in shape.

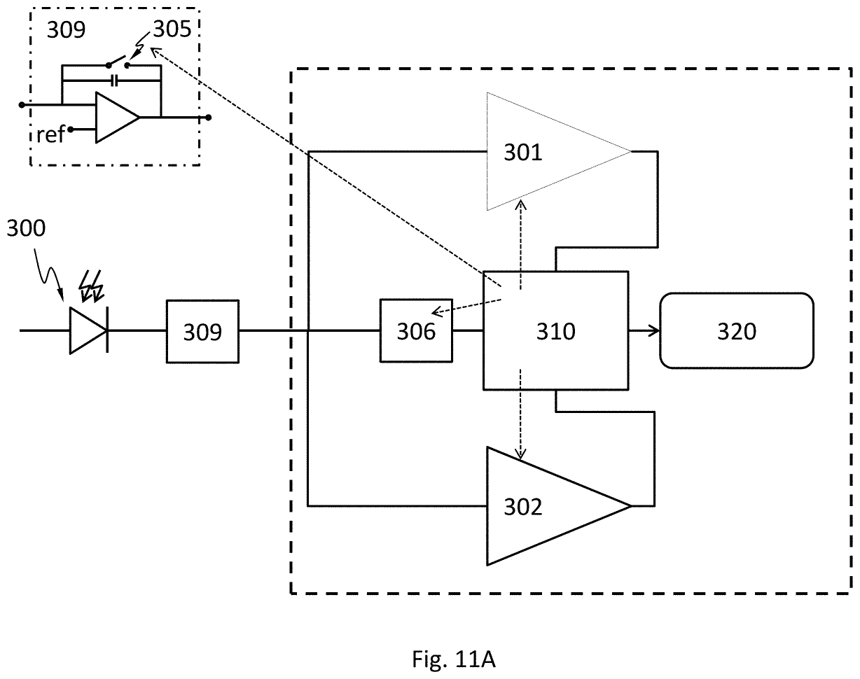

FIG. 10 schematically shows a flowchart of a method, according to an embodiment. In procedure 1010, the image sensor 9000 is positioned at the first position 14A relative to the object 50. In procedure 1020, the first set 19A of images of the object 50 is captured, by using the radiation detectors 100 of the image sensor 9000 and with the beam 13 of radiation from the radiation source 12, while moving the radiation source 12 among the first plurality of the positions on the path 15, relative to the object 50. In procedure 1030, the image sensor 9000 is positioned at the second position relative to the object 50. In procedure 1040, the second set 19B of images of the object 50 is captured, by using the radiation detectors 100 of the image sensor 9000 and with the beam 13 of radiation from the radiation source 12, while moving the radiation source 12 among the second plurality of positions on the path 15, relative to the object 50. Here, capturing the image “while” the radiation source 12 moves along the path 15 does not imply that the radiation source 12 is in motion relative to the object 50 when the image sensor 9000 captures an image. Instead, the radiation source 12 may be still relative to the object 50 when the image sensor 9000 captures an image, then move to the next position on the path 15, and then remain still at that next position when the image sensor 9000 captures the next image. In optional procedure 1050, at least one image in the first set 19A and at least one image in the second set 19B are stitched. In optional procedure 1060, a three-dimensional structure of the object 50 is determined based on the first set 19A of images or the second set 19B of images.

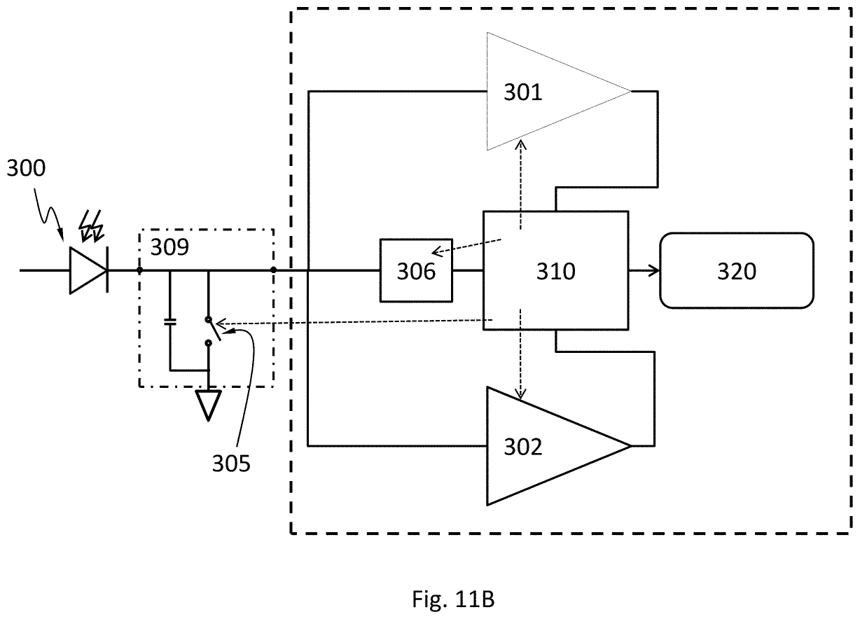

FIG. 11A and FIG. 11B each show a component diagram of the electronic system 121, according to an embodiment. The electronic system 121 may include a first voltage comparator 301, a second voltage comparator 302, a counter 320, a switch 305, a voltmeter 306 and a controller 310.

The first voltage comparator 301 is configured to compare the voltage of an electrode of a diode 300 to a first threshold. The diode may be a diode formed by the first doped region 111, one of the discrete regions 114 of the second doped region 113, and the optional intrinsic region 112. Alternatively, the first voltage comparator 301 is configured to compare the voltage of an electrical contact (e.g., a discrete portion of electrical contact 119B) to a first threshold. The first voltage comparator 301 may be configured to monitor the voltage directly or calculate the voltage by integrating an electric current flowing through the diode or electrical contact over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 configured as a continuous comparator reduces the chance that the system 121 misses signals generated by an incident particle of radiation. The first voltage comparator 301 configured as a continuous comparator is especially suitable when the incident radiation intensity is relatively high. The first voltage comparator 301 may be a clocked comparator, which has the benefit of lower power consumption. The first voltage comparator 301 configured as a clocked comparator may cause the system 121 to miss signals generated by some incident particles of radiation. When the incident radiation intensity is low, the chance of missing an incident particle of radiation is low because the time interval between two successive particles is relatively long. Therefore, the first voltage comparator 301 configured as a clocked comparator is especially suitable when the incident radiation intensity is relatively low. The first threshold may be 5-10%, 10%-20%, 20-30%, 30-40% or 40-50% of the maximum voltage one incident particle of radiation may generate in the diode or the resistor. The maximum voltage may depend on the energy of the incident particle of radiation (i.e., the wavelength of the incident radiation), the material of the radiation absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electrical contact over a period of time. The second voltage comparator 302 may be a continuous comparator. The second voltage comparator 302 may be controllably activated or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” |x| of a real number x is the non-negative value of x without regard to its signs. Namely,

$$\vert x \vert = \{ \begin{matrix}x , if x \geq 0 \\ - x , if x \leq 0.\end{matrix}$$

The second threshold may be 200%-300% of the first threshold. The second threshold may be at least 50% of the maximum voltage one incident particle of radiation may generate in the diode or resistor. For example, the second threshold may be 100 mV, 150 mV, 200 mV, 250 mV or 300 mV. The second voltage comparator 302 and the first voltage comparator 301 may be the same component. Namely, the system 121 may have one voltage comparator that can compare a voltage with two different thresholds at different times.

The first voltage comparator 301 or the second voltage comparator 302 may include one or more op-amps or any other suitable circuitry. The first voltage comparator 301 or the second voltage comparator 302 may have a high speed to allow the system 121 to operate under a high flux of incident radiation. However, having a high speed is often at the cost of power consumption.

The counter 320 is configured to register a number of particles of radiation reaching the diode or resistor. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electrical contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In an embodiment, the controller 310 is configured to activate the second voltage comparator at the beginning of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cutting off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause the number registered by the counter 320 to increase by one, if, during the time delay, the second voltage comparator 302 determines that the absolute value of the voltage equals or exceeds the absolute value of the second threshold.

The controller 310 may be configured to cause the voltmeter 306 to measure the voltage upon expiration of the time delay. The controller 310 may be configured to connect the electrode to an electrical ground, so as to reset the voltage and discharge any charge carriers accumulated on the electrode. In an embodiment, the electrode is connected to an electrical ground after the expiration of the time delay. In an embodiment, the electrode is connected to an electrical ground for a finite reset time period. The controller 310 may connect the electrode to the electrical ground by controlling the switch 305. The switch may be a transistor such as a field-effect transistor (FET).

In an embodiment, the system 121 has no analog filter network (e.g., a RC network). In an embodiment, the system 121 has no analog circuitry.

The voltmeter 306 may feed the voltage it measures to the controller 310 as an analog or digital signal.

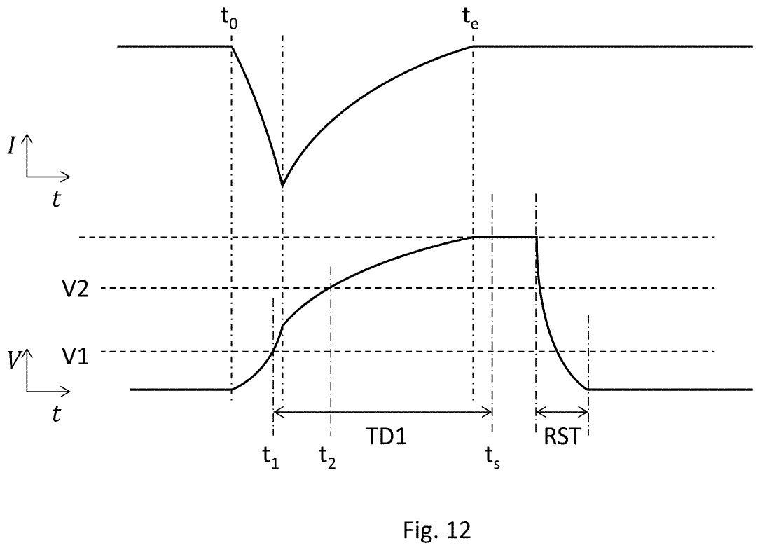

The system 121 may include an integrator 309 electrically connected to the electrode of the diode 300 or which electrical contact, wherein the integrator is configured to collect charge carriers from the electrode. The integrator can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electrode accumulate on the capacitor over a period of time (“integration period”) (e.g., as shown in FIG. 12, between t₀ to t₁, or t₁-t₂). After the integration period has expired, the capacitor voltage is sampled and then reset by a reset switch. The integrator can include a capacitor directly connected to the electrode.

FIG. 12 schematically shows a temporal change of the electric current flowing through the electrode (upper curve) caused by charge carriers generated by a particle of radiation incident on the diode or the resistor, and a corresponding temporal change of the voltage of the electrode (lower curve). The voltage may be an integral of the electric current with respect to time. At time t₀, the particle of radiation hits the diode or the resistor, charge carriers start being generated in the diode or the resistor, electric current starts to flow through the electrode of the diode or the resistor, and the absolute value of the voltage of the electrode or electrical contact starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 causes the number registered by the counter 320 to increase by one. At time tₑ, all charge carriers generated by the particle of radiation drift out of the radiation absorption layer 110. At time tₛ, the time delay TD1 expires. In the example of FIG. 12, time tₛ is after time tₑ; namely TD1 expires after all charge carriers generated by the particle of radiation drift out of the radiation absorption layer 110. The rate of change of the voltage is thus substantially zero at tₛ. The controller 310 may be configured to deactivate the second voltage comparator 302 at expiration of TD1 or at t₂, or any time in between.

The controller 310 may be configured to cause the voltmeter 306 to measure the voltage upon expiration of the time delay TD1. In an embodiment, the controller 310 causes the voltmeter 306 to measure the voltage after the rate of change of the voltage becomes substantially zero after the expiration of the time delay TD1. The voltage at this moment is proportional to the amount of charge carriers generated by a particle of radiation, which relates to the energy of the particle of radiation. The controller 310 may be configured to determine the energy of the particle of radiation based on voltage the voltmeter 306 measures. One way to determine the energy is by binning the voltage. The counter 320 may have a sub-counter for each bin. When the controller 310 determines that the energy of the particle of radiation falls in a bin, the controller 310 may cause the number registered in the sub-counter for that bin to increase by one. Therefore, the system 121 may be able to detect a radiation image and may be able to resolve particle of radiation energies of each particle of radiation.

After TD1 expires, the controller 310 connects the electrode to an electric ground for a reset period RST to allow charge carriers accumulated on the electrode to flow to the ground and reset the voltage. After RST, the system 121 is ready to detect another incident particle of radiation. Implicitly, the rate of incident particles of radiation the system 121 can handle in the example of FIG. 12 is limited by 1/(TD1+RST). If the first voltage comparator 301 has been deactivated, the controller 310 can activate it at any time before RST expires. If the controller 310 has been deactivated, it may be activated before RST expires.

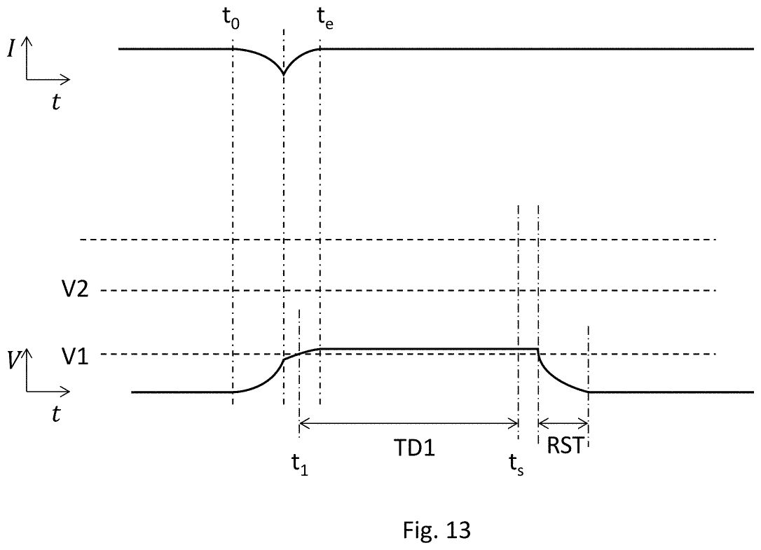

FIG. 13 schematically shows a temporal change of the electric current flowing through the electrode (upper curve) caused by noise (e.g., dark current, background radiation, scattered radiations, fluorescent radiations, shared charges from adjacent pixels), and a corresponding temporal change of the voltage of the electrode (lower curve), in the system 121 operating in the way shown in FIG. 12. At time t₀, the noise begins. If the noise is not large enough to cause the absolute value of the voltage to exceed the absolute value of V1, the controller 310 does not activate the second voltage comparator 302. If the noise is large enough to cause the absolute value of the voltage to exceed the absolute value of V1 at time t₁ as determined by the first voltage comparator 301, the controller 310 starts the time delay TD1 and the controller 310 may deactivate the first voltage comparator 301 at the beginning of TD1. During TD1 (e.g., at expiration of TD1), the controller 310 activates the second voltage comparator 302. The noise is very unlikely large enough to cause the absolute value of the voltage to exceed the absolute value of V2 during TD1. Therefore, the controller 310 does not cause the number registered by the counter 320 to increase. At time tₑ, the noise ends. At time tₛ, the time delay TD1 expires. The controller 310 may be configured to deactivate the second voltage comparator 302 at expiration of TD1. The controller 310 may be configured not to cause the voltmeter 306 to measure the voltage if the absolute value of the voltage does not exceed the absolute value of V2 during TD1. After TD1 expires, the controller 310 connects the electrode to an electric ground for a reset period RST to allow charge carriers accumulated on the electrode as a result of the noise to flow to the ground and reset the voltage. Therefore, the system 121 may be very effective in noise rejection.

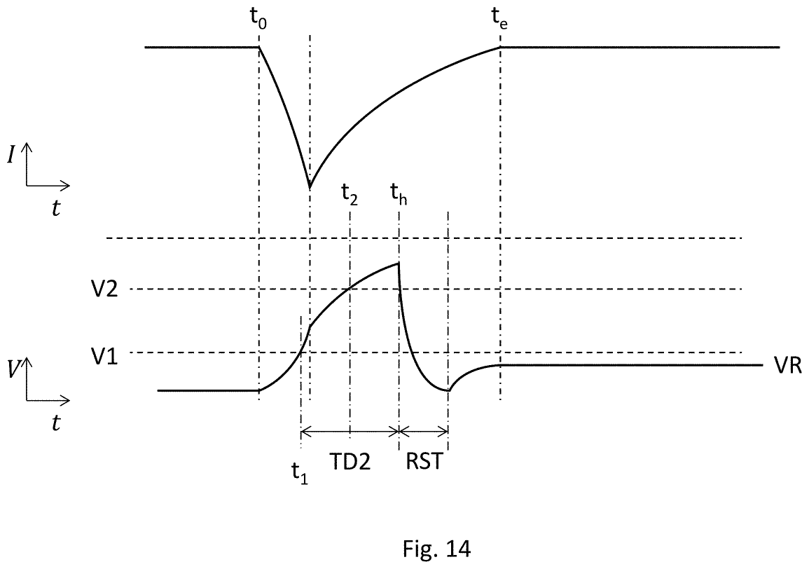

FIG. 14 schematically shows a temporal change of the electric current flowing through the electrode (upper curve) caused by charge carriers generated by a particle of radiation incident on the diode or the resistor, and a corresponding temporal change of the voltage of the electrode (lower curve), when the system 121 operates to detect incident particles of radiation at a rate higher than 1/(TD1+RST). The voltage may be an integral of the electric current with respect to time. At time t₀, the particle of radiation hits the diode or the resistor, charge carriers start being generated in the diode or the resistor, electric current starts to flow through the electrode of the diode or the electrical contact of resistor, and the absolute value of the voltage of the electrode or the electrical contact starts to increase. At time t₁, the first voltage comparator 301 determines that the absolute value of the voltage equals or exceeds the absolute value of the first threshold V1, and the controller 310 starts a time delay TD2 shorter than TD1, and the controller 310 may deactivate the first voltage comparator 301 at the beginning of TD2. If the controller 310 is deactivated before t₁, the controller 310 is activated at t₁. During TD2 (e.g., at expiration of TD2), the controller 310 activates the second voltage comparator 302. If during TD2, the second voltage comparator 302 determines that the absolute value of the voltage equals or exceeds the absolute value of the second threshold at time t₂, the controller 310 causes the number registered by the counter 320 to increase by one. At time tₑ, all charge carriers generated by the particle of radiation drift out of the radiation absorption layer 110. At time tₕ, the time delay TD2 expires. In the example of FIG. 14, time tₕ is before time tₑ; namely TD2 expires before all charge carriers generated by the particle of radiation drift out of the radiation absorption layer 110. The rate of change of the voltage is thus substantially non-zero at tₕ. The controller 310 may be configured to deactivate the second voltage comparator 302 at expiration of TD2 or at t₂, or any time in between.

The controller 310 may be configured to extrapolate the voltage at tₑ from the voltage as a function of time during TD2 and use the extrapolated voltage to determine the energy of the particle of radiation.

After TD2 expires, the controller 310 connects the electrode to an electric ground for a reset period RST to allow charge carriers accumulated on the electrode to flow to the ground and reset the voltage. In an embodiment, RST expires before tₑ. The rate of change of the voltage after RST may be substantially non-zero because all charge carriers generated by the particle of radiation have not drifted out of the radiation absorption layer 110 upon expiration of RST before tₑ. The rate of change of the voltage becomes substantially zero after tₑ and the voltage stabilized to a residue voltage VR after tₑ. In an embodiment, RST expires at or after tₑ, and the rate of change of the voltage after RST may be substantially zero because all charge carriers generated by the particle of radiation drift out of the radiation absorption layer 110 at tₑ. After RST, the system 121 is ready to detect another incident particle of radiation. If the first voltage comparator 301 has been deactivated, the controller 310 can activate it at any time before RST expires. If the controller 310 has been deactivated, it may be activated before RST expires.

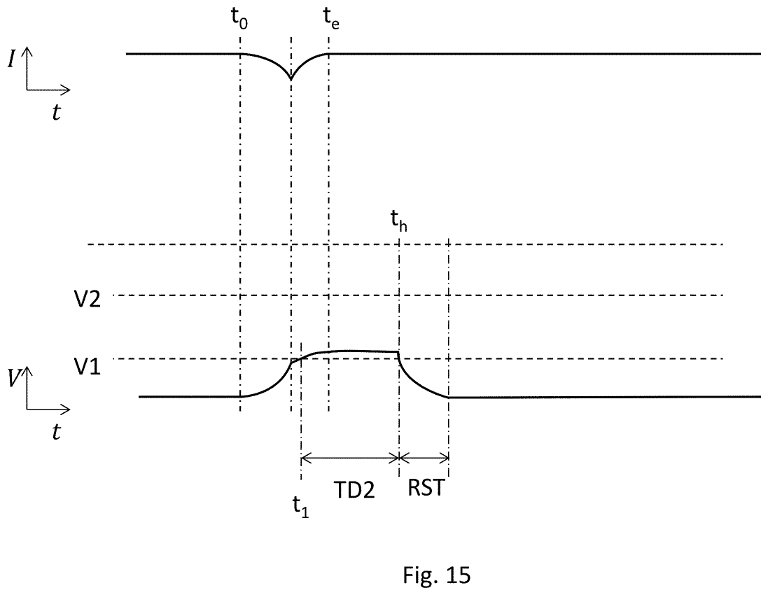

FIG. 15 schematically shows a temporal change of the electric current flowing through the electrode (upper curve) caused by noise (e.g., dark current, background radiation, scattered radiations, fluorescent radiations, shared charges from adjacent pixels), and a corresponding temporal change of the voltage of the electrode (lower curve), in the system 121 operating in the way shown in FIG. 14. At time to, the noise begins. If the noise is not large enough to cause the absolute value of the voltage to exceed the absolute value of V1, the controller 310 does not activate the second voltage comparator 302. If the noise is large enough to cause the absolute value of the voltage to exceed the absolute value of V1 at time t₁ as determined by the first voltage comparator 301, the controller 310 starts the time delay TD2 and the controller 310 may deactivate the first voltage comparator 301 at the beginning of TD2. During TD2 (e.g., at expiration of TD2), the controller 310 activates the second voltage comparator 302. The noise is very unlikely large enough to cause the absolute value of the voltage to exceed the absolute value of V2 during TD2. Therefore, the controller 310 does not cause the number registered by the counter 320 to increase. At time tₑ, the noise ends. At time tₕ, the time delay TD2 expires. The controller 310 may be configured to deactivate the second voltage comparator 302 at expiration of TD2. After TD2 expires, the controller 310 connects the electrode to an electric ground for a reset period RST to allow charge carriers accumulated on the electrode as a result of the noise to flow to the ground and reset the voltage. Therefore, the system 121 may be very effective in noise rejection.

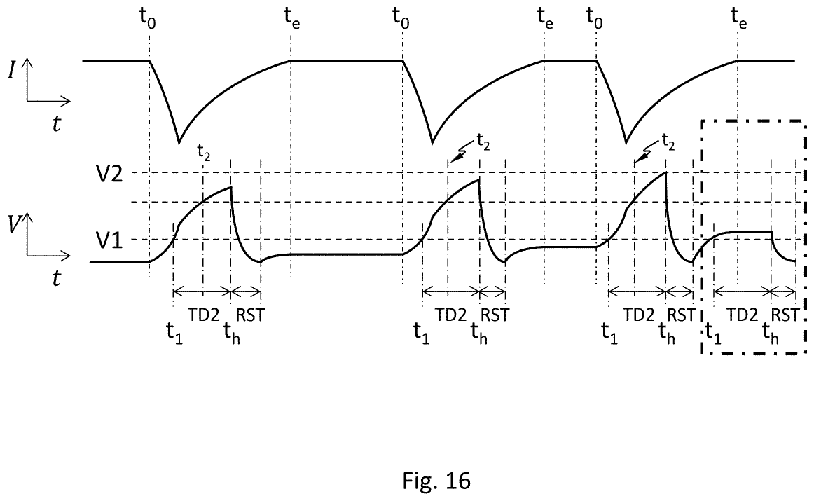

FIG. 16 schematically shows a temporal change of the electric current flowing through the electrode (upper curve) caused by charge carriers generated by a series of particles of radiation incident on the diode or the resistor, and a corresponding temporal change of the voltage of the electrode (lower curve), in the system 121 operating in the way shown in FIG. 14 with RST expires before tₑ. The voltage curve caused by charge carriers generated by each incident particle of radiation is offset by the residue voltage before that particle. The absolute value of the residue voltage successively increases with each incident particle. When the absolute value of the residue voltage exceeds V1 (see the dotted rectangle in FIG. 16), the controller starts the time delay TD2 and the controller 310 may deactivate the first voltage comparator 301 at the beginning of TD2. If no other particle of radiation incidence on the diode or the resistor during TD2, the controller connects the electrode to the electrical ground during the reset time period RST at the end of TD2, thereby resetting the residue voltage. The residue voltage thus does not cause an increase of the number registered by the counter 320.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An apparatus comprising:
a radiation source configured to produce a beam of radiation toward an object;
an image sensor comprising a plurality of radiation detectors spaced apart from one another;
wherein the image sensor is configured to move between a first position and a second position, relative to the object;
wherein the radiation source is configured to move along a path relative to the object.

2. The apparatus of claim 1,
wherein the image sensor is configured to capture a first set of images of the object, by using the radiation detectors and with the beam of radiation, while the image sensor is at the first position and the radiation source is respectively at a first plurality of positions on the path;
wherein the image sensor is configured to capture a second set of images of the object, by using the radiation detectors and with the beam of radiation, while the image sensor is at the second position and the radiation source is respectively at a second plurality of positions on the path.

3. The apparatus of claim 2, wherein the first plurality of positions on the path and the second plurality of positions on the path are the same.

4. The apparatus of claim 2, further comprising a processor configured to stitch at least one image in the first set and at least one image in the second set.

5. The apparatus of claim 2, further comprising a processor configured to determine a three-dimensional structure of the object based on the first set of images or the second set of images.

6. The apparatus of claim 1, wherein the object is a breast of a human.

7. The apparatus of claim 1, wherein the radiation source is configured to rotate with respect to the object, while the radiation source moves along the path.

8. The apparatus of claim 1, wherein the path is an arc around the object.

9. The apparatus of claim 1, wherein the image sensor comprises a collimator with a plurality of radiation transmitting zones and a radiation blocking zone;
wherein the radiation blocking zone is configured to block radiation that would otherwise incident on a dead zone of the image sensor, and the radiation transmitting zones are configured to transmit at least a portion of radiation that would incident on active areas of the image sensor.

10. (canceled)

11. (canceled)

12. (canceled)

13. (canceled)

14. The apparatus of claim 1, wherein the beam of radiation is a divergent beam of radiation.

15. The apparatus of claim 1, wherein the radiation is X-ray.

16. (canceled)

17. (canceled)

18. (canceled)

19. (canceled)

20. (canceled)

21. (canceled)

22. (canceled)

23. (canceled)

24. A method comprising:
positioning an image sensor at a first position relative to an object, the image sensor comprising a plurality of radiation detectors spaced apart from one another;
capturing a first set of images of the object, by using the radiation detectors and with a beam of radiation from a radiation source, while moving the radiation source among a first plurality of positions on a path, relative to the object;
positioning the image sensor at a second position relative to the object;
capturing a second set of images of the object, by using the radiation detectors and with the beam of radiation, while moving the radiation source among a second plurality of positions on the path, relative to the object.

25. The method of claim 24, wherein the first plurality of positions on the path and the second plurality of positions on the path are the same.

26. The method of claim 24, further comprising stitching at least one image in the first set and at least one image in the second set.

27. The method of claim 24, further comprising determining a three-dimensional structure of the object based on the first set of images or the second set of images.

28. The method of claim 24, wherein the object is a breast of a human.

29. The method of claim 24, wherein moving the radiation source comprises rotating the radiation source with respect to the object.

30. The method of claim 24, wherein the path is an arc around the object.

31. The method of claim 24, wherein the image sensor comprises a collimator with a plurality of radiation transmitting zones and a radiation blocking zone;
wherein the radiation blocking zone is configured to block radiation that would otherwise incident on a dead zone of the image sensor, and the radiation transmitting zones are configured to transmit at least a portion of radiation that would incident on active areas of the image sensor.

32. (canceled)

33. (canceled)

34. (canceled)

35. (canceled)

36. The method of claim 24, wherein the beam of radiation is a divergent beam of radiation.

37. The method of claim 24, wherein the radiation is X-ray.

38. (canceled)

39. (canceled)

40. (canceled)

41. (canceled)

42. (canceled)

43. (canceled)

44. (canceled)

45. (canceled)
